(19) United States
(12) Patent Application Publication
(10) Pub. No.: US 2023/0010088 A1
(43) Pub. Date: Jan. 12, 2023

(54) Arthroscopic Pump Saline Management

(71) Applicant: Medos International Sarl, Le Locle (CH)

(72) Inventors: Oliver J. Dumas, East Sandwich, MA (US); Eduard Grinberg, Sharon, MA (US); Michael Confoey, Westwood, MA (US); Pedro J. Miguel, Fall River, MA (US); Garrett Goode, Brighton, MA (US)

(21) Appl. No.: 17/370,688

(22) Filed: Jul. 8, 2021

(51) Int. Cl. A61M 1/00

(52) U.S. Cl. CPC A61M 1/777; A61M 2205/3334; A61M 2205/75; A61M 2205/50

(57) ABSTRACT

In general, devices, systems, and methods for arthroscopic pump saline management are provided. In an exemplary embodiment, a pump system including a pump is configured to manage fluid pumped by the pump to a surgical site during performance of a surgical procedure. The pump system being configured to manage fluid pumped to the surgical site can allow the pump system to manage fluid pressure at the surgical site by monitoring fluid loss at the surgical site.

### FIELD

The present disclosure generally relates to arthroscopic pump saline management.

### BACKGROUND

Arthroscopic pumps are used in a variety of surgical procedures in connection with a variety of functions, such as soft tissue ablation, contouring, cutting, coagulation, and temperature control. During performance of a surgical procedure an arthroscopic pump may provide irrigation (inflow) of fluid such as saline to a surgical site, e.g., a joint of a patient, and aspiration (outflow) of fluid from the surgical site. The pump may control fluid pressure at the joint to help provide joint distension for easy access, maintain good visibility, and/or to control bleeding. However, as a length of the surgical procedure increases, soft tissue at the surgical site becomes lax and increasingly leaks saline due to the sustained pressure at the joint. Thus, more saline has to be provided to the surgical site to account for the saline leakage as the length of the surgical procedure increases. More saline use increases cost of a surgical procedure since more saline has to be purchased for use during a surgical procedure. The increased costs compound for a hospital or other purchaser over multiple surgical procedures where more and more saline is used the longer a surgical procedure lasts.

It can also be challenging for nurses or other medical personnel to replace saline bags during a surgical procedure to ensure adequate saline supply because there are many other tasks the nurses or other medical personnel are also responsible for performing during the surgical procedure.

Additionally, less saline is typically needed later in a surgical procedure because less tissue cutting is being performed than earlier in the surgical procedure and, thus, the chances of bleeding are less later in the surgical procedure. Increasing saline delivery to a surgical site later in the surgical procedure may thus be excessive and unnecessary to control bleeding.

Accordingly, there remains a need for improved devices, systems, and methods for arthroscopic pumps.

### SUMMARY

In general, devices, systems, and methods for arthroscopic pump saline management are provided.

In one aspect, a surgical system is provided that in one embodiment includes an arthroscopic pump configured to pump fluid to a joint during performance of a surgical procedure to regulate fluid pressure at the joint to a stored set-point. The surgical system also includes a processor configured to control the pumping of the fluid to the joint, estimate fluid loss at the joint, and adjust, in real time with the performance of the surgical procedure, the set-point according to the estimated fluid loss.

The surgical system can have any number of variations. For example, estimating the fluid loss can include monitoring a flow rate of the fluid pumped to the surgical site over a period of time during the performance of the surgical procedure, and the adjusting can occur after the period of time such that the fluid is pumped to the surgical site according to the set-point during the first period of time and according to the adjusted set-point after the period of time. For another example, the pump can be configured to pump fluid from the joint during the performance of the surgical procedure, and estimating the fluid loss can include subtracting a flow rate of the fluid pumped from the joint from a flow rate of the fluid pumped to the joint. For yet another example, the estimation of the fluid loss can be over a plurality of minutes during the performance of the surgical procedure, the fluid can be pumped to the joint according to the set-point during the plurality of minutes, the adjusting can occur after the plurality of minutes has passed, and the fluid can be pumped to the joint according to the adjusted set-point after the plurality of minutes has passed. For another example, the pump can include a memory configured to store the set-point therein, and the pump can include the processor. For still another example, the fluid can be saline.

In another aspect, a surgical method is provided that in one embodiment includes pumping fluid to a surgical site during performance of a surgical procedure to regulate fluid pressure at the surgical site to a pressure set-point, estimating fluid loss at the surgical site, and adjusting, in real time with the performance of the surgical procedure, the pressure set-point according to the estimated fluid loss.

The surgical method can vary in any number of ways. For example, estimating the fluid loss can include monitoring a flow rate of the fluid pumped to the surgical site over a period of time during the performance of the surgical procedure, and the adjusting can occur after the period of time such that the fluid is pumped to the surgical site according to the pressure set-point during the first period of time and according to the adjusted pressure set-point after the period of time. For another example, pumping the fluid to the surgical site according to the adjusted pressure set-point can gradually reduce fluid pressure at the surgical site.

For yet another example, the surgical method can also include pumping fluid from the surgical site during the performance of the surgical procedure, and estimating the fluid loss can include subtracting a flow rate of the fluid pumped from the surgical site from a flow rate of the fluid pumped to the surgical site. In some embodiments, estimating the fluid loss can also include filtering fluid flow rate measurements indicative of the flow rate affected by an external suction source.

For still another example, the estimation of the fluid loss can be over a plurality of minutes during the performance of the surgical procedure, the fluid can be pumped to the surgical site according to the pressure set-point during the plurality of minutes, the adjusting can occur after the plurality of minutes has passed, and the fluid can be pumped to the surgical site according to the adjusted pressure set-point after the plurality of minutes has passed. For another example, an irrigation pump can pump the fluid to the surgical site, and a processor can control the pumping of the fluid to the surgical site and can perform the estimating and the adjusting. For yet another example, an arthroscopic pump can pump the fluid to the surgical site and can pump the fluid from the surgical site, the surgical site can include a joint, and the fluid can be saline.

In another embodiment, a surgical method includes pumping fluid to a joint during performance of a surgical procedure according to a joint pressure set-point, monitoring fluid leakage from the joint during the performance of the surgical procedure, and, based on the monitoring, changing the joint pressure set-point during the performance of the surgical procedure and pumping fluid to the joint during the performance of the surgical procedure according to the changed joint pressure set-point.

The surgical method can have any number of variations. For example, pumping the fluid to the joint during the performance of the surgical procedure according to the changed joint pressure set-point can gradually reduce fluid pressure at the joint and can control fluid leakage from the joint. In some embodiments, the surgical method can also include setting at least one of a rate of the gradual fluid pressure reduction and a minimum fluid pressure limit.

For another example, the surgical method can also include pumping fluid from the joint during the performance of the surgical procedure, and changing the joint pressure set-point can include subtracting a flow rate of the fluid pumped from the joint from a flow rate of the fluid pumped to the joint. For yet another example, the surgical method can also include determining a baseline portal leakage during the performance of the surgical procedure. For another example, an irrigation pump can pump the fluid to the joint, and a processor can control the pumping of the fluid to the joint and can perform the monitoring and the changing. For yet another example, the fluid can be saline.

### BRIEF DESCRIPTION OF DRAWINGS

This disclosure will be more fully understood from the following detailed description taken in conjunction with the accompanying drawings, in which:

FIG. 1 is a perspective view of one embodiment of an arthroscopic fluid pump;

FIG. 2 is a block diagram of the pump of FIG. 1 operatively coupled to a surgical site via inflow tubing, sheath, and outflow tubing;

FIG. 3 is a state diagram of one embodiment of fluid management of the pump of FIG. 1;

FIG. 4 is a flowchart of one embodiment of a process of fluid management;

FIG. 5 is a graph showing joint pressure versus surgical time and portal leakage flow versus surgical time; and

FIG. 6 is a flowchart of another embodiment of a process of fluid management.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

A person skilled in the art will appreciate that a time may not be precisely at a time but nevertheless be considered to be about that time due to any number of factors, such as sensitivity of measurement equipment. A person skilled in the art will appreciate that a value may not be precisely at a value but nevertheless be considered to be substantially at that value due to any number of factors, such as sensitivity of measurement equipment.

In general, devices, systems, and methods for arthroscopic pump saline management are provided. In an exemplary embodiment, a pump system including a pump is configured to manage fluid pumped by the pump to a surgical site during performance of a surgical procedure. The pump system is configured to maintain fluid pressure at the surgical site. However, sustaining the fluid pressure at the surgical site will, over time, cause soft tissue to stretch, or become lax, and thus cause increased leaking of the fluid. More and more fluid will thus leak the longer the surgical procedure lasts, thereby requiring more and more fluid to be pumped to the surgical site to maintain fluid pressure. A length of a surgical procedure in which fluid is pumped to the surgical site varies but can last at least about twenty minutes and last about three hours, and in some situations can last longer than about three hours. After about twenty minutes to about forty minutes, the soft tissue has typically distended enough to exacerbate fluid leakage. The pump system being configured to manage fluid pumped to the surgical site can allow the pump system to manage fluid pressure at the surgical site by monitoring fluid loss, which is also referred to herein as portal loss, at the surgical site. In other words, the pump system can be configured to monitor fluid leakage caused by stretched soft tissue at the portal.

Monitoring fluid loss can allow the pump system to reduce the fluid pressure being maintained at the surgical site as fluid loss increases over the course of the surgical procedure. Lower fluid pressure corresponds to less fluid being pumped to maintain that fluid pressure. Less fluid may thus be used in a surgical procedure since less fluid can be pumped to the surgical site over a total length of the surgical procedure, thereby saving cost of saline per surgical procedure. Fewer fluid supply bags (or other supply containers) may be needed in a surgical procedure because less fluid is needed over the total length of the surgical procedure, thereby saving nurses and/or other medical personnel the time of replacing used or partially used supply bags (or other supply containers) during the surgical procedure. Saline is typically not needed to control bleeding later in surgical procedures as much as earlier in surgical procedures because most or all tissue cutting tends to occur in the early stages of a surgical procedure, so reducing saline delivery to a surgical site later in a surgical procedure will not adversely affect bleeding control.

In an exemplary embodiment, the devices, systems, and methods of pump fluid management described herein are used in an arthroscopic surgical procedure context with an arthroscopic pump configured to measure fluid pressure at the pump and/or estimate fluid pressure at a joint. Although the pump fluid management described herein is discussed with respect to arthroscopic pumps and arthroscopic use, the pump fluid management described herein can be used in non-arthroscopic surgical procedures and with other types of pumps.

Additionally, the pump fluid management described herein can be implemented to manage saline pumped to a surgical site, other types of fluid pumped to a surgical site can be similarly managed.

One example of an arthroscopic pump is the FMS VUE® II available from Depuy Mitek of Raynham, Mass. Various embodiments of arthroscopic pumps, various embodiments of joint pressure estimation, and various embodiments of tissue shavers that can be used with a pump are further described in U.S. Pat. No. 10,874,776 entitled “Methods, Systems, And Devices For Joint To Pump Elevation Level User Interfaces, Autocalibration For Joint Elevation, And Joint Pressure Estimation” issued Dec. 29, 2020, U.S. Pat. No. 9,782,193 entitled “Tissue Shaving Device Having A Fluid Removal Path” issued Oct. 10, 2017, U.S. Pat. No. 9,186,166 entitled “Tissue Shavers” issued Nov. 17, 2015, and U.S. Pat. Pub. No. 2017/0120039 entitled “Anti-Clogging Fluid Management System” published May 4, 2017, which are hereby incorporated by reference in their entireties.

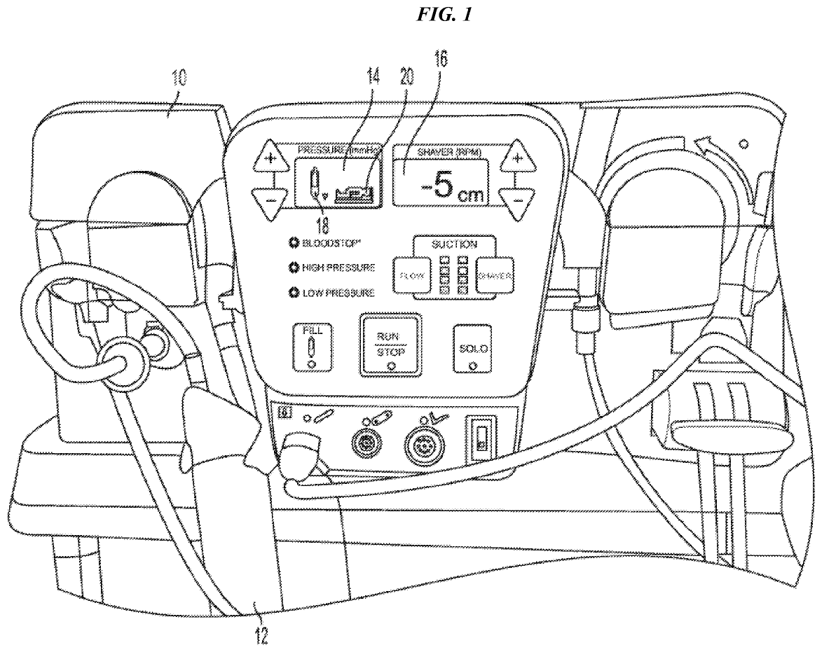

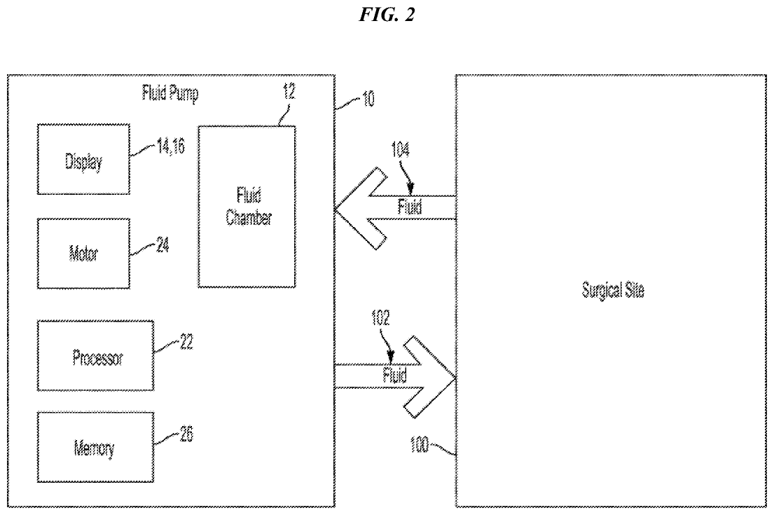

FIGS. 1 and 2 illustrate one embodiment of a pump system including an arthroscopic pump 10 configured to manage fluid pumped by the pump 10 to a surgical site 100. In an exemplary embodiment the surgical site 100 is at a joint such as the knee or shoulder.

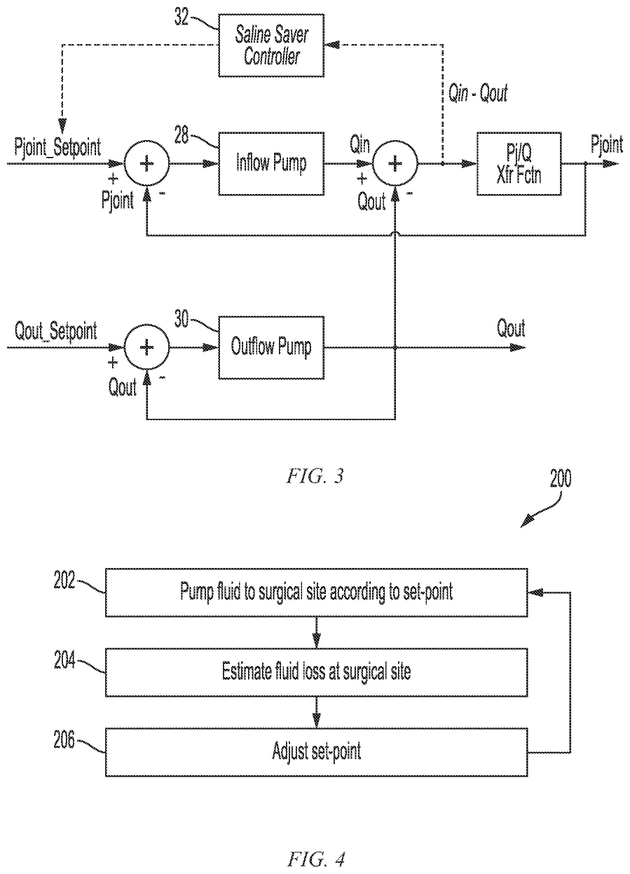

The pump 10 can have a variety of configurations. In this illustrated embodiment, as also shown in FIG. 3, the pump 10 includes an irrigation pump 28, also referred to herein as an “inflow pump,” configured to pump fluid to the surgical site 100, and the pump 10 includes an aspiration pump 30, also referred to herein as an “outflow pump,” configured to pump fluid from the surgical site 100. In an exemplary embodiment the fluid is saline but, as mentioned above, can be another fluid. The pump 10 includes joint inflow tubing 102 to allow fluid to flow between the pump 10 and the surgical site. FIG. 2 schematically illustrates the pump 10 operatively connected to the surgical site 100 via the inflow tubing 102 and illustrates outflow tubing 104 which returns to the aspiration pump 30 and then to a waste reservoir. The pump 10 also includes a fill chamber or reservoir 12 containing fluid therein to be pumped to the surgical site 100. The chamber 12 can, as in this illustrated embodiment, be used to smooth the fluid flow and to provide pressure sensing with a sensing tube at the top of the reservoir 12. Pressure is sensed at the fluid level in the chamber 12 so the fluid level is the effective pressure sensor location.

The pump 10 in this illustrated embodiment is configured to estimate fluid pressure at the surgical site 100. As mentioned above, in an exemplary embodiment the surgical site 100 is at a joint such that the pump 10 is configured to estimate fluid pressure at a joint.

The pump system also includes a processor 22 that is configured to control the irrigation pump and the aspiration pump. The pump 10 is configured to measure fluid pressure at the pump 10 based on fluid pressure within the reservoir 12 and on pump motor speed, e.g., a speed of a motor 24 configured to drive the pump 10. The pump 10 is configured to adjust the pressure measured at the pump 10, as controlled by the processor 22, to determine estimated pressure at the surgical site 100 using one or more control algorithms. The one or more control algorithms are stored in a memory 26 of the pump system and are executable by the processor 22. The processor 22 and the memory 26 are shown as part of the pump 10 in FIG. 2, but in other embodiments the processor 22 and/or the memory 26 can be located elsewhere in the pump system. For example, a computer system, e.g., a laptop computer, desktop computer, a tablet computer, a server, etc., configured to communicate (wired and/or wirelessly) with the pump 10 can include the processor 22 and/or the memory 26.

The pump system also includes a user interface configured to facilitate user interaction with the pump 10. The user interface includes a first display 14 configured to display joint pressure (in mmHg in this illustrated embodiment) in real time with use of the pump 10 during performance of a surgical procedure. The fluid pressure shown on the first display 14 is the estimated pressure of fluid at the surgical site. The user interface also includes a second display 16 configured to display speed (in revolutions per minute (RPM) in this illustrated embodiment) of a shaver operably coupled to the pump 10. The second display 16 in this illustrated embodiment is also configured to show a pump fill chamber icon 18 and a patient icon 20 configured to be in one of three positions relative to the pump fill chamber icon 18 to indicate whether the surgical site 100 is elevated above the fill chamber 12 (patient icon 20 in an upper position), the surgical site 100 is at a same elevation as the fill chamber 12 (patient icon 20 in a middle or neutral position), or the surgical site 100 is lower than the fill chamber 12 (patient icon 20 in a lower position).

The pump 10 in this illustrated embodiment is configured to estimate the fluid pressure at the surgical site 100. The estimation is calculated in real time with performance of the surgical procedure. The estimation can be calculated in a variety of ways. In this illustrated embodiment, the estimated fluid pressure at the surgical site 100 is based on at least one of elevation difference between the pump 10 and the surgical site 100 and tubing through which fluid flows between the pump 10 and the surgical site 100. In an exemplary embodiment the pump 10 is configured to estimate pressure based on each of these two factors, although pressure may be estimated using only one of these factors. Each of the inflow tubing 102 and the outflow tubing 104 have an associated sheath, in which case the fluid pressure can be estimated based on the tubing 102 and its associated sheath.

FIG. 4 illustrates one embodiment of a process 200 of managing fluid pumped by a pump to a surgical site. The process 200 is described with respect to the pump 10 of FIGS. 1-3, but other pumps can be similarly used. Also, the process 200 is described with respect to the surgical site 100 being at a joint, but other surgical sites are possible.

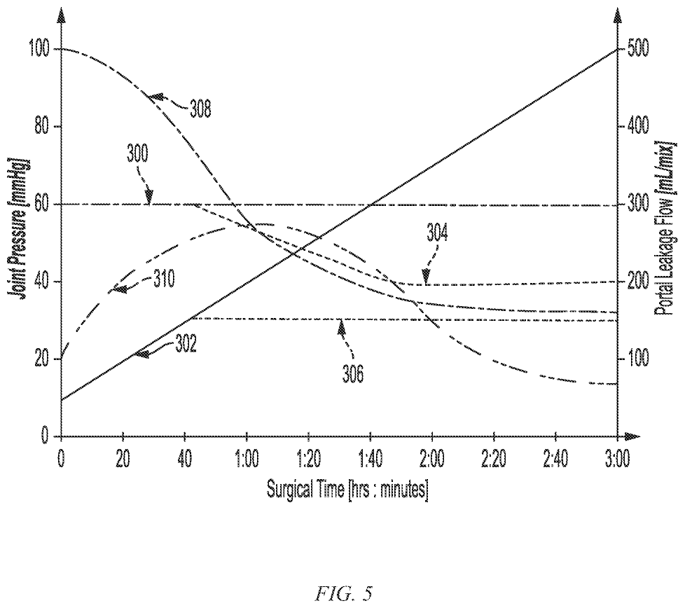

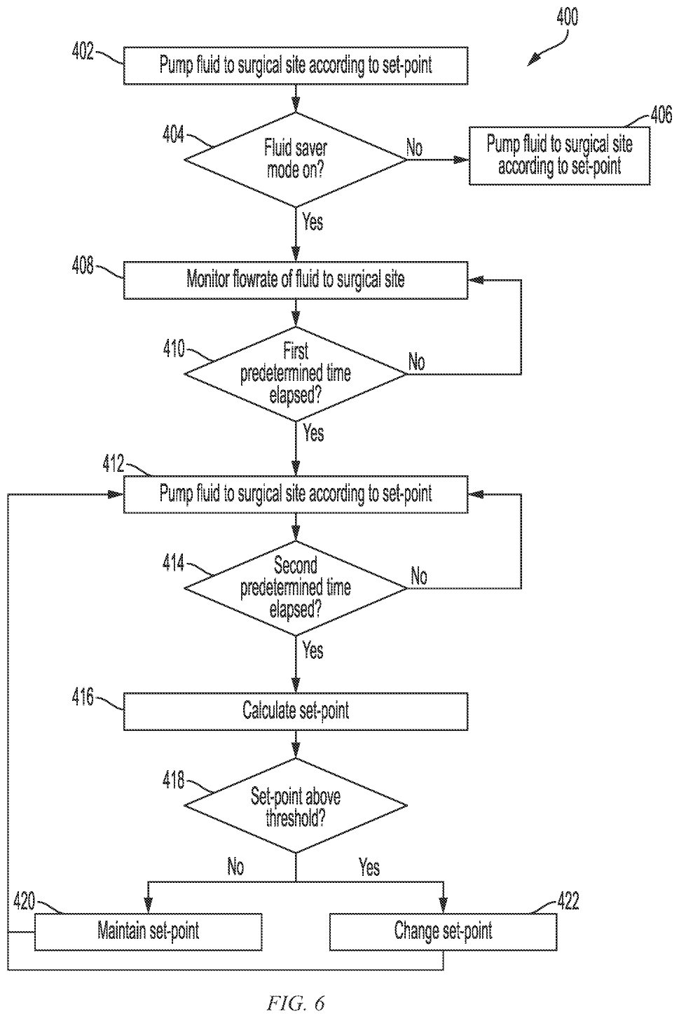

In the process 200, the pump 10, e.g., the inflow pump 28, pumps 202 fluid to the surgical site 100 through the inflow tubing 102 according to a set-point 300 for pressure at the surgical site 100. The set-point 300 in this illustrated embodiment is 60 mmHg, as shown in a graph of FIG. 5. The set-point 300 reflects a target pressure for the surgical site 100. The set-point 300 can be a non-changeable, pre-programmed value that is stored in the memory 26. Alternatively, the set-point 300 can be an adjustable value that a surgeon or other user can input to the pump 10 for a particular surgical procedure, e.g., via the pump's user interface using the pressure+/−arrow buttons and the run/stop button, before the pump 10 begins pumping fluid to the surgical site 100. The input set-point can then be stored in the memory 26.

With the pump 10 pumping 202 the fluid to the surgical site 100, the pump 10, e.g., the processor 22 thereof, estimates 204 fluid loss at the surgical site 100. The graph of FIG. 5 shows a line 302 indicating portal leakage flow (in mL/min) over surgical time (hours:minutes), where surgical time reflects length of time the pump is pumping fluid in the surgical procedure. In an exemplary embodiment, estimating 204 the fluid loss can include monitoring a flow rate of the fluid pumped to the surgical site 100. Embodiments of monitoring flow rate are discussed further below.

With the pump 10 pumping 202 the fluid to the surgical site 100, the pump 10, e.g., the processor 22 thereof, adjusts 206 the set-point. The adjustment 206 occurs in real time with performance of the surgical procedure in which the pump 10 is being used, with the pump 10 pumping 202 the fluid to the surgical site 100, and with the pump 10, e.g., the outflow pump 30, pumping fluid from the surgical site 100. The adjustment 206 is based on the estimated 204 fluid loss at the surgical site 100. In general, the pump 10, e.g., the processor 22 thereof, uses the estimate 204 fluid loss to adjust 206 the set-point to a lower pressure value. Embodiments of adjusting a set-point are discussed further below. Reducing the set-point allows less fluid to be pumped to the surgical site 100 since a lower pressure need be maintained at the surgical site 100.

After the set-point adjustment 206, the pump 10 continues pumping 202 the fluid to the surgical site 100 according to the set-point, which has now been adjusted to a lower pressure value. The graph of FIG. 5 shows a dotted line 304 indicating that the pressure at the surgical site 100 decreases from about forty minutes lapsed surgical time (0:40), which is when the set-point adjustment 206 first occurred in the surgical procedure. This decrease is due to the set-point adjustment 206 and the pump's pumping 202 of fluid according to the adjusted 206 set-point. Were the set-point not adjusted 206 and then used in controlling fluid pumping to the surgical site 100, the joint pressure would remain at the original set-point 300 (60 mmHg in this illustrated embodiment). The graph of FIG. 5 also shows a dotted line 306 indicating post-set-point adjustment portal leakage flow over surgical time. The portal leakage flow 306 is substantially constant once the adjusted 206 set-point begins being used at about forty minutes lapsed surgical time. The portal leakage flow 306 is substantially constant at about 150 mL/min in this illustrated embodiment, but the post-set-point adjustment portal leakage flow 306 may be different in another surgical procedure. Were the set-point not adjusted 206 and then used as adjusted in controlling fluid pumping to the surgical site 100, the portal leakage flow would not be substantially constant (line 306) but would instead continue to increase (line 302) as the surgical procedure continued over time.

As mentioned above, the pump 10 in this illustrated embodiment is configured to estimate fluid pressure at the surgical site 100. The graph of FIG. 5 also shows a line 308 of pressure versus surgical time and a line 310 of portal leakage flow versus surgical time for a pump that does not estimate fluid pressure at a surgical site but instead measures fluid pressure at the pump. Pump pressure does not equal surgical site pressure because of elevation, tubing, or sheath losses. From time zero (start of the pump pumping fluid), the leakage flow line 310 increases as tissue softens and more portal loss occurs, so the pressure line 308 decreases. The lowering pressure eventually begins to cause less leakage over time, at about time 1:10 in the graph of FIG. 5, since the tissue is softening less and/or has reached peak softening. In other words, as reflected by line 310, the portal leakage flow is naturally corrected when using such a pump because the pressure naturally decreases over time due to pressure being measured at the pump, which does not account for elevation, tubing, or sheath losses. While joint pressure can decrease over time with such a pump, the pressure is unknown to the surgeon and/or other medical professionals performing the surgical procedure, and the pressure can fall below a desired minimum pressure, thereby making the surgical procedure more challenging to perform. FIG. 5 shows this undesirable drop below a desired minimum pressure of about 200 mmHg occurring for the pump (line 310) at about time 1:45. The desired minimum pressure is about 40 mmHg in this illustrated example but can be another value, e.g., a value in a range of about 20 mmHg to about 40 mmHg such as about 40 mmHg, about 35 mmHg, about 30 mmHg, about 25 mmHg, about 20 mmHg, or another value. Conversely, the dotted line 304 of pressure versus time for the pump 10 in which the set-point is adjusted shows that the pressure does not ever fall below the desired minimum pressure of about 40 mmHg, as will be discussed further below. Additionally, the dotted line 304 of pressure versus time for the pump 10 shows that the desired minimum pressure is reached at about time 1:45, whereas the line 310 of pressure versus time for the other pump shows that the desired minimum pressure is reached earlier, at about time 1:30. The pump 10 may thus allow for higher pressure at the surgical site 100 for longer than the other pump can achieve.

FIG. 6 illustrates one embodiment of a process 400 of managing fluid pumped by a pump to a surgical site. The process 400 is described with respect to the pump 10 of FIGS. 1-3, but other pumps can be similarly used. Also, the process 400 is described with respect to the surgical site 100 being at a joint, but other surgical sites are possible.

In the process 200, the pump 10, e.g., the inflow pump 28, pumps 402 fluid to the surgical site 100 through the inflow tubing 102 according to a set-point for pressure at the surgical site 100. As mentioned above, the set-point can be stored in the pump's memory 26 and can be a preset value or an adjustable value. The set-point for pumping fluid to the surgical site 100 is represented by variable Pjoint_Setpoint in FIG. 3. The joint pressure is represented by variable Pjoint in FIG. 3.

The process 400 includes determining 402 whether the pump 10 is in a fluid saver mode. The pump 10 in this illustrated embodiment has two modes of fluid management. In a first, non-fluid saver mode, the pump 10 uses the same set-point in managing fluid flow throughout the pump's use in a surgical procedure. In the non-fluid saver mode, the set-point is not adjusted after the set-point has been set (either preset or set by a user). In a second, fluid saver mode, the pump 10 can adjust the set-point during use of the pump in a surgical procedure, e.g., in real time with the pump 10 pumping fluid to the surgical site 100. The pump 10 in the second mode may thus use a different set-point at different times during the surgical procedure. In other embodiments, a pump can only include the fluid saver mode. In such embodiments, the process 400 does not include determining 404 whether the fluid saver mode is on.

The pump 10 can prompt a surgeon or other user to select, e.g., via the pump's user interface, the non-fluid saver mode or the fluid saver mode. The surgeon or other user may decide for the pump 10 to operate in the non-fluid saver mode if, for example, the surgical procedure in which the pump 10 will be used is expected to be relatively short and thus not a procedure in which portal leakage will cause much or any adverse effect. In general, portal leakage tends to begin causing an adverse effect, e.g., requiring increased saline use, causing distension, causing reduced visibility, etc., after about thirty minutes to about sixty minutes. The surgeon or other user may therefore decide, for example, to select non-fluid saver mode for a surgical procedure expected to be less than about thirty minutes, less than about forty minutes, less than about fifty minutes, or less than about sixty minutes. Using the graph of FIG. 5 by way of example, the surgical procedure may be expected to be about forty minutes or less, in which case there would be no difference in fluid management between the fluid saver mode and the non-fluid saver mode. The surgeon or other user may decide for the pump 10 to operate in the fluid saver mode if, for example, the surgical procedure in which the pump 10 will be used is expected to be relatively long or, for another example, the surgeon or other user prefers a conservative approach of enabling the fluid saver mode in case the surgical procedure lasts longer than expected. In an exemplary embodiment, the mode prompt is provided before the pump 10 begins pumping 402 the fluid. The mode prompt can, however, be provided at some point between the pump beginning to pump 402 the fluid and a first predetermined amount of time (discussed further below).

If the pump 10 is determined 404 to be in the non-fluid saver mode, e.g., by checking a mode flag stored in the memory 26, the pump 10 pumps 406 fluid according to the preset set-point throughout the pump's use in the surgical procedure. If the pump 10 is in the fluid saver mode, the pump 10 monitors 408 a flow rate of fluid to the surgical site 100. In general, the monitoring 408 estimated fluid loss at the surgical site. In an exemplary embodiment, the monitoring 408 of the flow rate of fluid to the surgical site 100 includes measuring the flow rate at a predetermined frequency, e.g., every five seconds, every ten seconds, every fifteen seconds, every twenty seconds, every twenty-five seconds, every thirty seconds, or at another frequency, for a first predetermined amount of time since the start of the pumping 402, e.g., a time elapsed from time zero of the graph of FIG. 5. Measuring the flow rate includes subtracting a flow rate of the fluid pumped from the surgical site 100 (represented by Qin in FIG. 3) from a flow rate of the fluid pumped to the surgical site 100 (represented by Qout in FIG. 3). In general, the first predetermined amount of time is an amount of time in which portal leakage has likely not started to cause an adverse effect. For example, the first predetermined amount of time can be in a range of about ten minutes to about fifteen minutes, e.g., ten minutes, eleven minutes, twelve minutes, thirteen minutes, fourteen minutes, fifteen minutes, or another time amount, although other first predetermined amounts of time are possible. The pump 10 can be configured to omit any abruptly changed flow rate measurements, which are indicative of flow rate being affected by an external suction source (e.g., an RF electrode, a shaver with external suction, etc.), using a filter that filters out such measurements.

The monitoring 408 continues as the pump 10 continues pumping 402 fluid until the pump 10 determines 410 that the first predetermined amount of time has elapsed, e.g., using a timer or counter in communication with the pump's processor 22. An average of the monitored 408 flow rates, e.g., average of each (Qin−Qout) measurement, which can be calculated by the pump 10, e.g., by the processor 22 thereof, represents a base portal leakage value. The average will likely be different in different surgical procedures because of one or more factors such as the particular tissue to which the fluid is being pumped, initial set-point, and/or other factor(s).

When the first predetermined amount of time has been determined 410 to have elapsed, the pump 10 continues to pump 412 fluid according to the set-point until a second predetermined amount of time has been determined 414 to have elapsed. For example, if the first predetermined amount of time is about forty minutes as in this illustrated embodiment, the pump 412 continues pumping 412 fluid until the second predetermined amount of time has elapsed from time 0:40 of the graph of FIG. 5. In general, the second predetermined amount of time defines how often the set-point is adjusted. In an exemplary embodiment, the second predetermined amount of time is a non-zero time that is less than about three minutes, e.g., about three minutes, about 2.5 minutes, about two minutes, about 1.5 minutes, about one minute, about thirty seconds, about twenty seconds, or about another amount of time.

When the second predetermined amount of time has been determined 414 to have elapsed, the pump 10 calculates 416 an adjusted set-point. Calculating 416 the set-point includes comparing a current measured portal leakage with the base portal leakage. The pump 10 thus measures a flow rate of fluid to the surgical site 100 when the second predetermined amount of time has been determined 414 to have elapsed to identify a current measured portal leakage. If the current measured portal leakage is greater than the base portal leakage plus a leakage offset, then the pump 10 reduces the set-point by a predetermined decrease amount to achieve the adjusted set-point. The predetermined decrease amount can be, for example, 1 mmHg, 0.5 mmHg, 2 mmHg, or another amount. The leakage offset is a preset value that helps ensure the portal leakage does not exceed the base portal leakage by a set value. In an exemplary embodiment the leakage offset is a value in a range of 30 mL/min to 50 mL/min. In this illustrated embodiment, the leakage offset is 40 mmHg. In some embodiments, the leakage offset can be time dependent and can change over time based on desired fluid usage profile over time. For example, the leakage offset can be reduced at a constant rate over time as less pressure is needed at the surgical site 100 the longer the surgical procedure lasts. If the current measured portal leakage is less than the base portal leakage plus the leakage offset, then the pump 10 increases the set-point by a predetermined increase amount to achieve the adjusted set-point. The predetermined increase amount can be, for example, 1 mmHg, 0.5 mmHg, 2 mmHg, or another amount. In an exemplary embodiment, the predetermined increase amount is the same as the predetermined decrease amount, which may help maintain smooth pressure transitions substantially undetectable by a surgeon and/or other medical professionals performing the surgical procedure.

In some embodiments, the predetermined decrease and increase amounts are preset and non-adjustable. In other embodiments, the predetermined decrease and increase amounts can be adjustable by a user, such as by being input via the pump's user interface. A user may want different predetermined decrease and increase amounts in different surgical procedures based on surgeon preference, a type of surgical procedure being performed (e.g., meniscectomy, ACL repair, PCL repair, labrum surgery, rotator cuff surgery, bicep surgery, hip surgery, etc.), and/or other factors.

Before storing the adjusted set-point in the memory 26 as the new set-point to use in pumping fluid to the surgical site 100, the pump 10, e.g., the processor 22 thereof, determines 418 if the adjusted set-point is greater than a predetermined minimum pressure threshold. The predetermined minimum pressure threshold represents a minimum pressure below which the pump 10 should not maintain at the surgical site 100 because, e.g., it may result in adverse effects of too-low pressure. In this illustrated embodiment, the predetermined minimum pressure threshold is 40 mmHg, as reflected in the graph of FIG. 5 in which the dotted line 304 of pressure does not go below 40 mmHg. The portal leakage flow can thus also be prevented from falling below a minimum level, which is 200 mL/min in the illustrated example of FIG. 5. In some embodiments, the predetermined minimum pressure threshold is preset and non-adjustable. In other embodiments, the minimum pressure threshold can be adjustable by a user, such as by being input via the pump's user interface. A user may want different minimum pressure thresholds in different surgical procedures based on surgeon preference, a type of surgical procedure being performed (e.g., meniscectomy, ACL repair, PCL repair, labrum surgery, rotator cuff surgery, bicep surgery, hip surgery, etc.), and/or other factors.

If the adjusted set-point is not greater than the predetermined pressure threshold, then the pump 10 maintains 420 the set-point stored in the memory 26, and fluid continues being pumped 412 to the surgical site 100 according to the set-point already stored in the memory 26. In other words, the adjusted set-point is not saved in the memory 26 as the new set-point to use in pumping fluid. If the adjusted set-point is greater than the predetermined pressure threshold, then the pump 10 changes 422 the set-point stored in the memory 26 by saving the adjusted set-point in the memory 26 as the new set-point.

Fluid continues then being pumped 412 to the surgical site 100 according to the set-point stored in the memory 26, which is the newly determined adjusted set-point. Thus, as the set-point is adjusted repeatedly over time during performance of the surgical procedure and the fluid is pumped 412 to the surgical site according to the set-point, fluid pressure at the surgical site 100 can be gradually reduced, e.g., at a rate in a range of about 0.1 mmHg to about 0.2 mmHg/min, but does not fall below the predetermined minimum pressure threshold, as reflected in the graph of FIG. 5.

The process 400 continues until the pump 10 stops pumping fluid to the surgical site 100.

In some embodiments, instead of waiting the second predetermined amount of time when the first predetermined amount of time has been determined 410 to have elapsed, the pump 10 can adjust 416 the set-point without first determining 414 whether the second predetermined amount of time has elapsed. Thus, before adjusting 416 the set-point for the first time, there can be substantially zero time elapsed before the pump 10 adjusts 416 the set-point.

The process 400 is described with respect to managing a set-point for pump inflow, but a set-point for pump outflow can be similarly managed. The set-point for pumping fluid from the surgical site 100 is represented by variable Qout_Setpoint in FIG. 3 and would be the managed set-point for pump outflow.

In a dual pump system, e.g., a first pump for fluid outflow and a second pump for fluid outflow such as that shown in FIG. 3 including an inflow pump 28 and an outflow pump 30, pump fluid management can be implemented as discussed above. In other embodiments, an arthroscopic pump (or other type of pump) can be configured as a single pump system configured to pump fluid to a surgical site, e.g., include an irrigation pump, but not pump fluid from the surgical site. In such a single pump system, pump fluid management as discussed herein can be implemented with the assumption that outflow is zero. In other words, a flow rate of the fluid pumped to the surgical site (represented by Qout) is assumed to be zero.

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

1. A surgical system, comprising:
an arthroscopic pump configured to pump fluid to a joint during performance of a surgical procedure to regulate fluid pressure at the joint to a stored set-point; and
a processor configured to
control the pumping of the fluid to the joint,
estimate fluid loss at the joint, and
adjust, in real time with the performance of the surgical procedure, the set-point according to the estimated fluid loss.

2. The system of claim 1, wherein estimating the fluid loss includes monitoring a flow rate of the fluid pumped to the joint over a period of time during the performance of the surgical procedure; and
the adjusting occurs after the period of time such that the fluid is pumped to the joint according to the set-point during the first period of time and according to the adjusted set-point after the period of time.

3. The system of claim 1, wherein the pump is configured to pump fluid from the joint during the performance of the surgical procedure; and
estimating the fluid loss includes subtracting a flow rate of the fluid pumped from the joint from a flow rate of the fluid pumped to the joint.

4. The system of claim 1, wherein the estimation of the fluid loss is over a plurality of minutes during the performance of the surgical procedure;
the fluid is pumped to the joint according to the set-point during the plurality of minutes;
the adjusting occurs after the plurality of minutes has passed; and
the fluid is pumped to the joint according to the adjusted set-point after the plurality of minutes has passed.

5. The system of claim 1, wherein the pump includes a memory configured to store the set-point therein; and
the pump includes the processor.

6. The system of claim 1, wherein the fluid is saline.

7. A surgical method, comprising:
pumping fluid to a surgical site during performance of a surgical procedure to regulate fluid pressure at the surgical site to a pressure set-point;
estimating fluid loss at the surgical site; and
adjusting, in real time with the performance of the surgical procedure, the pressure set-point according to the estimated fluid loss.

8. The method of claim 7, wherein estimating the fluid loss includes monitoring a flow rate of the fluid pumped to the surgical site over a period of time during the performance of the surgical procedure; and
the adjusting occurs after the period of time such that the fluid is pumped to the surgical site according to the pressure set-point during the first period of time and according to the adjusted pressure set-point after the period of time.

9. The method of claim 7, wherein pumping the fluid to the surgical site according to the adjusted pressure set-point gradually reduces fluid pressure at the surgical site.

10. The method of claim 7, further comprising pumping fluid from the surgical site during the performance of the surgical procedure;
wherein estimating the fluid loss includes subtracting a flow rate of the fluid pumped from the surgical site from a flow rate of the fluid pumped to the surgical site.

11. The method of claim 10, wherein estimating the fluid loss also includes filtering fluid flow rate measurements indicative of the flow rate affected by an external suction source.

12. The method of claim 7, wherein the estimation of the fluid loss is over a plurality of minutes during the performance of the surgical procedure;
the fluid is pumped to the surgical site according to the pressure set-point during the plurality of minutes;
the adjusting occurs after the plurality of minutes has passed; and
the fluid is pumped to the surgical site according to the adjusted pressure set-point after the plurality of minutes has passed.

13. The method of claim 7, wherein an irrigation pump pumps the fluid to the surgical site; and
a processor controls the pumping of the fluid to the surgical site and performs the estimating and the adjusting.

14. The method of claim 7, wherein an arthroscopic pump pumps the fluid to the surgical site and pumps the fluid from the surgical site;
the surgical site includes a joint; and
the fluid is saline.

15. A surgical method, comprising:
pumping fluid to a joint during performance of a surgical procedure according to a joint pressure set-point;
monitoring fluid leakage from the joint during the performance of the surgical procedure; and
based on the monitoring, changing the joint pressure set-point during the performance of the surgical procedure and pumping fluid to the joint during the performance of the surgical procedure according to the changed joint pressure set-point.

16. The method of claim 15, wherein pumping the fluid to the joint during the performance of the surgical procedure according to the changed joint pressure set-point gradually reduces fluid pressure at the joint and controls fluid leakage from the joint.

17. The method of claim 16, further comprising setting at least one of a rate of the gradual fluid pressure reduction and a minimum fluid pressure limit.

18. The method of claim 15, further comprising pumping fluid from the joint during the performance of the surgical procedure;
wherein changing the joint pressure set-point includes subtracting a flow rate of the fluid pumped from the joint from a flow rate of the fluid pumped to the joint.

19. The method of claim 15, further comprising determining a baseline portal leakage during the performance of the surgical procedure.

20. The method of claim 15, wherein an irrigation pump pumps the fluid to the joint; and
a processor controls the pumping of the fluid to the joint and performs the monitoring and the changing.

21. The method of claim 15, wherein the fluid is saline.
